(19) United States
(12) Patent Application Publication
(10) Pub. No.: US 2022/0202285 A1
(43) Pub. Date: Jun. 30, 2022

(54) ACTIVE TEMPERATURE CONTROL SYSTEM FOR ANATOMIC SITES

(71) Applicant: GYRUS ACMI, INC. D/B/A OLYMPUS SURGICAL TECHNOLOGIES AMERICA, Westborough, MA (US)

(72) Inventors: Sergey A. Bukesov, Acton, MA (US); Kurt G. Shelton, Bedford, MA (US); Maria Rao, Worcester, MA (US); David Bloem, Maple Grove, MN (US)

(21) Appl. No.: 17/646,170

(22) Filed: Dec. 28, 2021

Related U.S. Application Data
(60) Provisional application No. 63/131,221, filed on Dec. 28, 2020.

(51) Int. Cl. A61B 1/12; A61B 34/00; A61B 18/14; A61B 18/20

(52) U.S. Cl. CPC A61B 1/128; A61B 34/76; A61B 2017/00115; A61B 18/20; A61B 18/14

(57) ABSTRACT

Systems, devices, and methods for temperature and pressure management of an anatomic site are provided. A system can include a scope configured to provide a view of an anatomic site, an energy delivery device configured to deliver therapy energy to the anatomic site, a first irrigation conduit configured to transfer fluid to the anatomic site, a first temperature sensor situated to provide first temperature data associated with the anatomic site, and control circuitry electrically coupled to receive the first temperature data, the control circuitry being configured to adjust, based at least in part on the first temperature data, at least one of (i) a first temperature of the fluid, (ii) a flow parameter of the fluid, or (iii) a setting of the energy delivery device to manage a second temperature of the anatomic site.

### RELATED APPLICATION

This application claims the benefit of priority to U.S. Provisional Patent Application No. 63/131,221 which was filed on Dec. 28, 2020 and titled “Active Cooling Irrigation System for Anatomic Sites”, which is incorporated by reference herein in its entirety.

### TECHNICAL FIELD

These teachings regard fluid temperature control for irrigation of an anatomic site.

### BACKGROUND

Endoscopic, arthroscopic, lithotripsy, and other scoped procedures typically include irrigation of fluid to clear a field of view of a scope from bodily fluids. Example bodily fluids include urine and blood. The fluid can scatter light and obscure an image provided by the scope. Scattered light makes it more difficult for a physician to clearly see a target anatomy (sometimes called an “anatomic site”). The irrigation fluid flow rate can be set high enough to keep the field of view of the endoscope clear of bodily fluids. This includes providing a pressurized fluid to the scope at the anatomic site. Such therapies can include laser, electrical power, or the like.

### SUMMARY

Teachings herein provide for temperature control of an anatomic site. Teachings provide an irrigation system, device, method, control system, or the like that can allow a physician to set a target anatomical temperature that is automatically managed. The system can maintain the temperature by monitoring one or more temperature sensor measurements and/or one or more flow rate measurements, and/or a therapy delivery power setting. The system can observe or measure the temperature rise and fall during the delivery of a therapy at a certain setting and at a certain irrigation temperature and flow rate. The system can use the observations or measurements to calculate the cooling power of the anatomic site and therefore the flow rate and temperature of fluid required to maintain the desired anatomical temperature. Various fluid temperatures can be achieved by mixing refrigerated fluid with room temperature fluid or even heated fluid.

A therapy delivery device can include or be coupled with a scope configured to provide a view of an anatomic site. The system can include an energy delivery device configured to deliver optical or electrical energy to the anatomic site. A first irrigation conduit can be situated to transfer fluid from a fluid reservoir to the anatomic site. A first temperature sensor can be situated to provide first temperature data associated with the anatomic site. Control circuitry can be electrically coupled to receive the first temperature data. The control circuitry can be configured to adjust, based on the first temperature data, at least one of (i) a first temperature of the fluid, (ii) a flow parameter of the fluid, or (iii) a setting of the energy delivery device to manage a second temperature of the anatomic site.

The system can include a suction device configured to remove fluid from the anatomic site resulting in removed fluid. A second conduit can be in fluid communication with the suction device to receive the removed fluid and configured to transfer the removed fluid away from the anatomic site. The first temperature data can include a third temperature of the removed fluid. The first temperature data can include the second temperature.

The system can include a fluid cooler in fluid communication with the fluid. The fluid cooler can be configured to receive and cool a first portion of the fluid resulting in cooled fluid. The control circuitry can adjust a fourth temperature to which the fluid cooler cools the cooled fluid based on the first temperature data. A third conduit can be in fluid communication with the fluid. The third conduit can be configured to receive a second portion of the fluid. The first conduit can be configured to receive a mixture of both the second portion of the fluid and the cooled fluid.

The system can include a fluid heater in fluid communication with the fluid. The fluid heater can be situated to receive and heat a third portion of the fluid resulting in heated fluid. The first conduit can be configured to receive a mixture of both the heated and cooled fluid.

An actuated valve can be in fluid communication with the first conduit and electrically coupled to the control circuitry. The actuated valve can be situated between the fluid cooler and the first conduit and situated between the fluid heater and the first conduit. The control circuitry can be configured to alter a physical state of the actuated valve based on the first temperature data.

A second temperature sensor can be situated to determine a fifth temperature of fluid out of the actuated valve. The control circuitry can be further configured to adjust respective temperature settings of the fluid heater and the fluid cooler based on the fifth temperature. The system can include a pump in fluid communication with the first conduit. The pump can be electrically coupled to the control circuitry. The control circuitry can be configured to adjust a pump rate of the pump based on the first temperature data.

A pressure sensor can be electrically coupled to the control circuitry and situated to generate pressure data representing pressure about the anatomic site. The control circuitry can be further configured to adjust a pump rate of the pump based on the pressure data. A flow sensor can be situated to determine a flow rate of fluid from the pump. The flow sensor can be electrically coupled to the control circuitry. The control circuitry can be further configured to adjust the rate of the pump based on the flow rate.

The control circuitry can be configured to adjust, based on the first temperature data, a setting of the energy delivery device to manage a temperature of the anatomic site. A display device can be electrically coupled to the control circuitry. The display device can be configured to provide a user a view of the first temperature. The user interface can be configured to receive data indicating a first temperature set point above which to maintain the anatomic site and a second temperature set point below which to maintain the anatomic site. The control circuitry can operate to manage the temperature of the anatomic site between first and second temperature set points.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1A illustrates, by way of example, a diagram of a system for management of fluid temperature of fluid provided to an anatomic site.

FIG. 1B illustrates, by way of example, a diagram of another system for management of fluid temperature of fluid provided to an anatomic site.

FIG. 2 illustrates, by way of example, a block diagram of another system for management of fluid temperature of the fluid provided to the anatomic site.

FIG. 3 illustrates, by way of example, a block diagram of another system for management of fluid temperature of the fluid provided to the anatomic site.

FIG. 4 illustrates, by way of example, a block diagram of a system for management of fluid temperature of a mixed fluid provided to the anatomic site.

FIG. 5 illustrates, by way of example, a block diagram of a system for management of fluid temperature of a mixed fluid provided to the anatomic site.

FIG. 6 illustrates, by way of example, a diagram of an embodiment of a method for temperature management of an anatomic site.

FIG. 7 illustrates, by way of example, a flow diagram of a method for determining a temperature, pressure, or a combination thereof, at the anatomic site.

FIG. 8 illustrates, by way of example, a diagram of a method for adjusting one or more components of the system of FIGS. 1A, 1B, 2, 3, 4, or 5 to regulate temperature at the anatomic site.

FIG. 9 illustrates, by way of example, a diagram of a method for adjusting one or more components of the system of FIGS. 1A, 1B, 2, 3, 4, or 5 to regulate pressure at the anatomic site.

FIG. 10 illustrates, by way of example, a block diagram of an embodiment of a machine (e.g., a computer system) to implement one or more embodiments.

### DETAILED DESCRIPTION

Endoscopic, arthroscopic, lithotripsy, and other scoped procedures may include irrigation of fluid to clear a field of view of a scope from bodily fluids. In such scoped procedures, this irrigation fluid is room temperature or heated saline. In addition to keeping the field of view clear, the fluid can also be used to cool the anatomic site. This cooling can help control a temperature of the anatomic site when a therapy being delivered causes heating of the anatomic site (and surrounding anatomy and fluids). Such therapies can include laser, electrical power, or the like. Delivering too much fluid to the anatomic site too quickly can lead to hypothermia in extreme cases, such as in highly vascular organs like the kidney. Delivering too little fluid to the anatomic site can lead to overheating of tissue or damage to tissue. In addition, delivering the fluid too slowly can cause the risk of fluid temperature remaining too high for an extended period of time. Therefore, the flow rate and starting temperature of the fluid, the duration of the procedure, or a combination thereof, have to be carefully considered during the procedure, thereby avoiding, or at least reducing the risk of hypothermia or overheating the healthy tissue.

To mitigate temperature concerns, a physician can use an irrigation system that provides active heating or cooling of the fluid. Body-temperature fluid is less effective at cooling than colder, actively cooled fluid. An anatomic site that is experiencing a rise in temperature due to a therapeutic treatment, such as laser lithotripsy, can benefit from fluid that is colder than body temperature.

A protein can start to denature at around 42° C. It can be considered undesirable for the temperature of any anatomy that is not the subject of therapeutic intervention to reach this temperature. In the case of using fluid at body temperature, a therapeutic intervention can only increase the local anatomical temperatures by about 5° C. before this limit is reached.

In the case of using room-temperature irrigation fluid, which is typically between 18° C. and 25° C., the therapeutic intervention can only increase the local anatomical temperatures by between 17° C.-24° C. before the same temperature limit of 42° C. is reached. Given a same average power of the therapeutic energy source (e.g., a laser control system, an electrical power supply, electrical generator, or the like that powers one or more lasers or electrodes), treatment can proceed for between 3 and 5 times longer using “room temperature fluid” as compared to body temperature fluid (assuming a linear relationship when using room temperature irrigation instead of body-temperature irrigation). For interventions where the therapy is an energy device, such as a laser or an electrosurgical system, actively cooled irrigation can better accommodate longer treatments with lower anatomical temperatures.

In the case of laser lithotripsy, the anatomical temperature increase is greater due to the heating power of the treatment. The power of the treatment causes overheating to occur quickly, even when using room temperature fluid. To allow for an increased continuous treatment time using laser lithotripsy, a cooled fluid source can be used. The increased continuous treatment times can reduce an overall time of a procedure by reducing an amount of time spent waiting for a target site to cool down. The increased treatment times can allow a physician to break, for example, a kidney stone into small enough parts using laser lithotripsy without having to worry about damaging tissue of the patient. The increased treatment times can allow a physician to continue to remove bad tissue with a monopolar or bipolar electrode. As used herein, cooled fluid means fluid is actively cooled beyond ambient cooling while room temperature fluid (sometimes called “direct fluid”) is not actively cooled and is only cooled by ambient temperature. Active cooling means using an electrical, gas or any suitable cooling technique to reduce the temperature of the fluid faster than setting the fluid in a room and waiting for the temperature to reduce. A cooled fluid from a cooled fluid source can increase treatment duration beyond times currently possible using direct fluid while still keeping the average temperature of the anatomical fluid in the vicinity of the treatment area below 42° C. and above hypothermic temperature levels.

Teachings herein provide for selective delivery of refrigerated (sometimes called “cooled”), heated, direct (fluid that is not subject to active heating or cooling), or a combination thereof, irrigation fluid to an anatomic site. For example, when anatomical temperatures are increasing above a user-programmable threshold (e.g., 34, 35, 36, 37, 38, 39, 40, 41, 42, 43, 44, 45C, or a greater or lesser temperature) cooled fluid can be provided to the anatomic site, such as through the scope or a conduit coupled to or used with the scope. The cooled fluid can be as cold as 4° C. but can be a greater temperature. In some configurations, the cooling rate of the cooled fluid can be greater than a corresponding warming rate of room temperature or warmer fluid, thus slowing or reversing the temperature increase at the anatomic site. As the measured temperature at the anatomic site starts to stabilize or decrease below a threshold, (e.g., 32, 31, 30, 29, 28° C., or a greater or lower temperature) the irrigation system can be configured to (e.g., automatically) deliver (e.g., more) direct fluid, heated fluid, or a combination thereof. The increase in the direct fluid or heated fluid can cause the anatomic site to warm, and the corresponding temperature of the anatomic site to increase. Hypothermia requires a body core temperature to fall below 35° C. This is not the same as measuring 35° C. at the treatment site since that temperature difference is only 2° C. below body temperature. A risk of hypothermia can be indicated by a specified amount of time below a temperature threshold. The amount of time at a specified temperature can be used to estimate what the temperature of the core of the body.

Teachings provide an irrigation system, device, method, control system, or the like that can allow a physician to set a target anatomical temperature that is automatically managed. The system can maintain the temperature by monitoring temperature sensor measurements, pressure sensor measurements, flow rate measurements, and/or settings of a therapy delivery device. The system can measure and/or compute the temperature rise and fall resulting from the delivery of a certain duration of therapy at a certain setting and at a certain irrigation temperature and flow rate and use the measurements/computation to determine the cooling rate (e.g., in degrees per unit time) of the anatomic site and therefore the flow rate and temperature of fluid required to achieve or maintain the desired anatomical temperature. The cooling rate is the amount a temperature is decreasing per unit time. A warming rate is the amount temperature is increasing per unit time. Both the cooling rate and the warming rate are examples of a more general temperature change rate. Various fluid temperatures can be achieved by mixing refrigerated fluid with room temperature fluid or heated fluid.

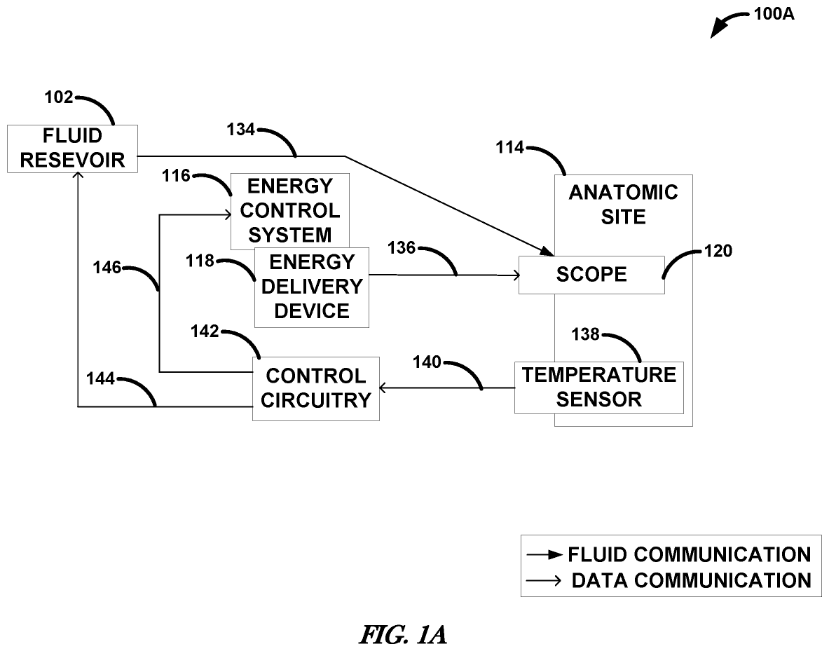

FIG. 1A illustrates, by way of example, a diagram of a system 100A for management of fluid temperature of fluid provided to an anatomic site 114. The system 100A as illustrated includes a fluid reservoir 102 fluidly communicating with the anatomic site 114. A conduit 134 is in fluid communication with the reservoir 102 and anatomic site 114. The conduit 134 receives the fluid from the reservoir 102 and transports the fluid to the anatomic site 114. An energy delivery scope (e.g., an endoscope) 120 is proximate or touching the anatomic site 114 for treatment. The scope 120 receives energy from an energy delivery device (e.g., a laser device, ultrasonic device, or electrical device) 118 via communication medium (e.g., an optical fiber, electrical conductor, or wireless communication medium) 136. The energy provided by the energy delivery device 118 is controlled by an energy control system (e.g., a laser energy generator, an electrical energy generator, an ultrasonic energy generator, or the like) 116. In one embodiment, a temperature sensor 138 is situated in or around the anatomic site 114. Additionally, or alternatively, the temperature sensor 138 can be situated to measure a temperature of fluid coming into the anatomic site 114 or fluid coming out of the anatomic site 114. The temperature sensor 138 provides temperature data, on electrical communication medium 140, to the control circuitry 142. The control circuitry 142 manages a temperature of the anatomic site 114 by modulating one or more parameters (e.g., temperature and/or flow rate) associated with delivery of the fluid, operating parameters (e.g., power, magnitude, frequency, voltage, current, duty cycle, energy level or the like) of the energy control system 116, or the like.

The fluid reservoir 102 can include a tap, bag, tank, bucket, or the like. The fluid from the fluid reservoir 102 (or another reservoir) can include saline or another fluid. The fluid can be gravity fed, titrated by a valve, or a combination thereof, out of the fluid reservoir 102 and into the conduit 134.

The conduit 134 is a hollow tube. A first end of the conduit 134 can be mechanically coupled to the reservoir 102 receive the fluid therefrom. An opposing, second end of the conduit 134 can be situated near the anatomic site 114. The conduit 134 can travel through the scope 120 or be permanently attached or detachably coupled to the scope 120. The conduit 134 transports the fluid to the anatomic site 114 while protecting the fluid from the environment about the conduit 134.

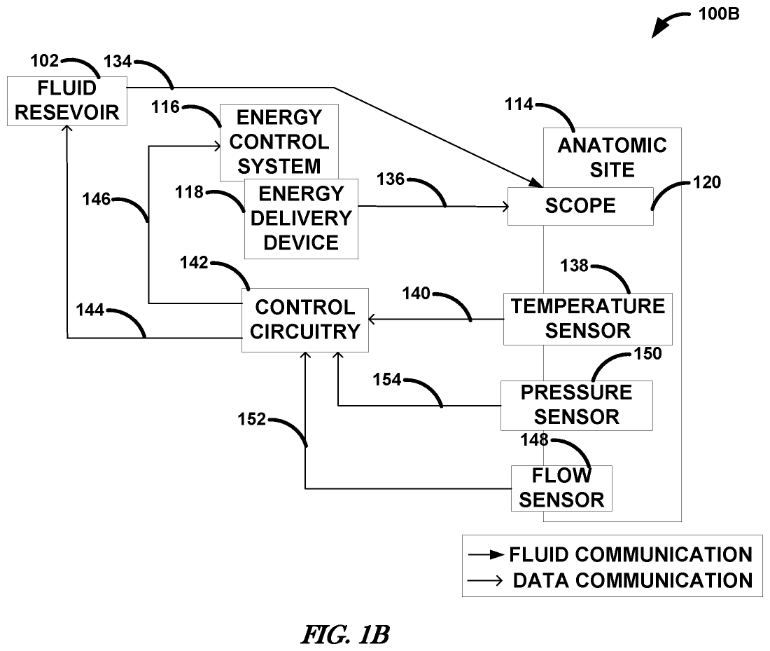

The scope 120 includes optical components (e.g., lenses, mirrors, collimators, filters, prisms, polarizers, beamsplitters, wave plates, fiber optics, a camara, or the like) configured to provide an image of the anatomic site 114. The scope 120 can include a communication medium (e.g., an optical fiber, electrical conductor, or wireless communication medium) 136 permanently or removably attached thereto. A physician, or other user, can view the anatomic site 114 through optics on the scope 120 or through a display 224 (see FIG. 2). The physician, or other user, can activate, de-activate, or modulate therapy provided by the energy delivery device 118 using a control knob, button, or other actuation mechanism on or coupled to, for example, a foot-actuated control pedal, and/or the scope 120. Additionally, or alternatively, the physician may control the energy delivery device 118 via a separate controlling system (e.g., a laser console touch screen display). The procedure of viewing inside a patient body is called an endoscopy. The scope 120 can be used to examine internal organs like a throat, sinus cavity, ureter, kidney, or esophagus. An endoscope can be specialized, such as to view a target organ. Such specialized endoscopes can be named after their target organ. For example, a sinuscope is specialized to provide a view of a sinus cavity, an otoscope is specialized to provide a view of an inner ear, a ureteroscope is specialized to provide a view of a ureter, a laryngoscope is specialized to provide a view of a larynx, a cystoscope is specialized to provide a view of the bladder, a nephroscope is specialized to view the kidney, a bronchoscope is specialized to view the bronchus, an arthroscope is specialized to view a joint, a colonoscope is specialized to view a colon, and a laparoscope is specialized to view an abdomen or pelvis.

The energy delivery device 118 can include a laser, an electrical power supply, or the like. The communication medium 136 can include an optic fiber (if the therapy device 118 includes a laser), an electrical conductor, such as can be coupled to a unipolar or bipolar electrode, (if the therapy device 118 includes an electrical power supply), or the like. The therapy device 118 can generate energy that is transferred, by the communication medium 136 to the anatomic site 114. The energy control system 116 can modulate the energy delivery device 118 so as to adjust the energy provided to the anatomic site 114 by the energy delivery device 118 and the communication medium 136. The energy control system 116 can modulate an operating parameter of the energy delivery device 118. Example operating parameters include an intensity, frequency, duration, or other parameter of laser therapy. Other example operating parameters include a magnitude, amplitude, frequency, shape, or other parameter of electrical therapy. Example energy control systems 116 include laser generators, electrical power generators, ultrasonic wave generators, or the like. These generators typically come with knobs, touchscreens, buttons, or the like that are user operable. A user can adjust a parameter of the output of the generator by providing input through the touchscreen, pressing a button, turning a knob, or the like. An energy control system 116 includes an input interface through which operating parameters can be adjusted. The input interface is electrically coupled with energy generating circuitry (or a controller of energy generating circuitry). The input interface adjusts the energy generating circuitry in accord with input received at the input interface or provides the input (in a same of different form as it was received) to the controller so the controller can cause the energy generating circuitry to be adjusted in accord with the input. The input can be provided by a user (turning a knob, touching a touchscreen, or the like), the control circuitry 142, a device controlled by the user (e.g., the scope 120, a foot pedal coupled to the scope 120, or the like) or a combination thereof.

The temperature sensor 138 determines a temperature or otherwise provides data that can be used to determine a temperature of the anatomic site 114. The temperature sensor 138 can include an infrared (IR) sensor, a thermocouple, a resistance temperature detector (RTD), a thermistor, a semiconductor based integrated circuit (IC), or the like. The temperature sensor 138 can be integrally formed with the scope 120, physically separate from the scope 120, attached to the scope 120, detachably coupled with the scope 120, or the like. When the energy delivery device 118 is a laser, an IR-based temperature sensor can be turned off while laser energy is supplied to the anatomic site 114. Temperature data from the temperature sensor 138 can be provided to the control circuitry 142 by communication medium 140. The temperature data can indicate a temperature associated with the anatomic site 114. For example, the scope 120 may include or be coupled to an IR-sensitive fiberoptic channel that can bring light back to an IR thermometer for measuring the temperature data. To avoid collision with the laser output emission, the thermal measurement may be switched on when the laser or other energy emission is off.

The control circuitry 142 includes electric or electronic components configured to provide control signals to the energy control system over communication medium 146 or to a fluid provisioning system over communication medium 144. The electric or electronic components can include one or more transistors, resistors, capacitors, diodes, inductors, oscillators, memory devices, amplifiers, analog to digital converters, digital to analog converters, multiplexers, switches, logic gates (e.g., AND, OR, XOR, negate, buffer, or the like), power supplies, processing units (e.g., a central processing unit (CPU), graphics processing unit (GPU), application specific integrated circuit (ASIC), field programmable gate array (FPGA), or the like), or the like.

The control circuitry 142 can reduce a power provided by the energy control system 116 if the temperature data satisfies a first criterion. The control circuitry 142 can communicate with the energy control system 116 over communication medium 146 (e.g., a wired or wireless communication medium). The control circuitry 142 can provide a control signal to the energy control system 116 that adjusts an energy delivery parameter of the energy generated by the energy control system 116.

The control circuitry 142 can increase a flow rate of the fluid from the fluid reservoir 102 if the temperature data satisfies the first criterion or a second, different criterion. The first criterion can include the temperature indicated by the temperature data being greater than or equal to a threshold temperature (e.g., a user-specified or default temperature). The second criterion can include a same or different temperature threshold.

The control circuitry 142 can increase a power provided by the energy control system 116 if the temperature data satisfies a third criterion. The control circuitry 142 can cause a flow rate of the fluid from the fluid reservoir 102 to decrease if the temperature data satisfies the third criterion or a fourth, different criterion. The third criterion can include the temperature indicated by the temperature data being less than or equal to a threshold temperature (e.g., a user-specified or default temperature). The fourth criterion can include a same or different temperature threshold. Any of the threshold temperatures can be set based on safe operating temperatures of the energy delivery device 118, and in one embodiment, can be set by the user. One or more of the threshold temperatures can be set to help ensure that the temperature of the tissue around the anatomic site 114 does not reach 42° C. Such a threshold temperature can include 34° C., 35° C., 36° C., 37, 38, 39, 40, 41, 42, 43, 44, 45° C., ora greater or lesser temperature that is less than 42° C. One or more of the threshold temperatures can be set to help ensure that the temperature of the tissue around the anatomic site 114 stays above a temperature corresponding to hypothermia. Hypothermia occurs as the body temperature drops below 35° C. The threshold lower temperature can be about 32, 31, 30, 29, 28° C., such as to help ensure that hypothermia does not occur during the duration of energy delivery.

Communication mediums 136, 140, 144, 146 (or other communication medium herein, such as in FIGS. 2, 3, 4, and 5) can include a wired, wireless, or optical communication mechanism. Wired communication mediums include conductors, such as traces, wires, or the like. Wireless communication mediums include a transmit antenna providing an electromagnetic transmission to a receive antenna. The electromagnetic transmission can conform to a communication protocol that defines how data is encoded on the electromagnetic transmission. Example wireless communication protocols include Bluetooth, Zigbee, WiFi, radio frequency identification (RFID), cellular, near field communication (NFC), or the like. Optical communication mediums include fiber optics, open air space, or the like, that allows light to travel therethrough in a constrained manner.

FIG. 1B illustrates, by way of example, a diagram of another system 100B that provides for temperature control at an anatomic site. The system 100B is similar to the system 100A, with the system 100B including an additional pressure sensor 150 and a flow sensor 148.

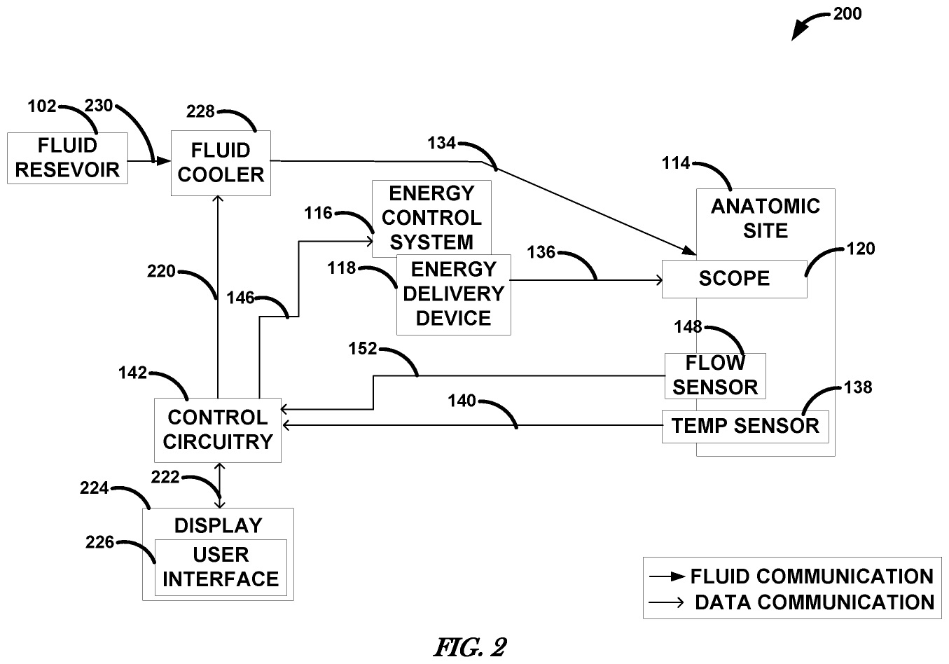

The flow sensor 150 can be on, or at least partially in, a suction device 330 (see FIG. 3), conduit 332, or the conduit 134. The flow sensor 336 can provide flow data to the control circuitry 142 through communication medium 152. The flow data can indicate how fast fluid and debris is being removed from or how much fluid, per unit time, is being provided to the anatomic site 114.

The pressure sensor 150 can provide the control circuitry 142 with pressure data over communication medium 154. The pressure data can indicate a fluid pressure near the anatomic site 114 and/or a pressure on tissue of the anatomic site 114. The pressure sensor 150 can be integrally formed with the scope 120, detachably coupled to the scope 120, attached to the scope 120, or physically separate from the scope 120. The pressure at the anatomic site 114 can indicate how much gas is being retained from fluid evaporation, how much fluid and debris is building up in the anatomic site 114, a temperature associated with the anatomic site 114, or a combination thereof. The pressure at the anatomic site 114 can affect the efficacy of the energy delivered to the anatomic site 114. By controlling the pressure, the control circuitry 142 can help ensure that the energy delivery is effective and safe.

The control circuitry 142 can determine a temperature associated with the anatomic site 114 based on the temperature data from the temperature sensor 138, the flow data from the flow sensor 150, pressure data from the pressure sensor 150, and/or any combination thereof. The control circuitry 142 can increase the flow rate of the fluid in the conduit 134, such as to alter the temperature at the anatomic site 114. The control circuitry 142 can alter the temperature set point of the fluid in the fluid reservoir 102 based on the temperature data from the temperature sensor 138, the flow data from the flow sensor 148, the pressure data from the pressure sensor 150, and/or any combination thereof.

Although FIGS. 1A and 1B depict that the temperature sensor 138, flow sensor 148 and/or pressure sensor 150 may be employed at or near the targeted anatomic site, the temperature sensor 138, flow sensor 148 and/or pressure sensor 150 may be employed at one or more sites that are not in proximity to the targeted anatomic site (e.g., on the proximal end of the scope 120).

The systems 100A, 100B help the user of the scope 120 retain a temperature of the anatomic site 114 (and anatomy around the anatomic site 114) below a de-naturing temperature and/or above a hypothermia-inducing temperature. The system 100A, 100B improves upon prior irrigation systems that do not include temperature, pressure, or flow rate data feedback to inform temperature management. The system 100A, 100B can selectively, adaptively, and smartly adjust the temperature at the target site based on the measured/computed temperature, flow rate, pressure, or a combination thereof.

FIG. 2 illustrates, by way of example, a block diagram of another system 200 for management of fluid temperature of the fluid from the fluid reservoir 102 provided to the anatomic site 114. The system 200 includes some components from the system 100A, 100B including the fluid reservoir 102, the conduit 134, the scope 120, the energy control system 116, the energy delivery device 118, the temperature sensor 138, the control circuitry 142, and the communication mediums 136, 140, and 146. The system 200 includes additional components including a fluid cooler 228, a display device 224, and communication mediums 220, 222.

A conduit 230 provides a path for the fluid to flow to the fluid cooler 228. The fluid cooler 228 reduces a temperature of the fluid. The fluid cooler 228 can operate by evaporating a coolant (e.g., turning a coolant from a liquid state to a gas state), thus cooling an area around the coolant. The fluid cooler 228 can operate based on the Peltier effect. Such coolers transfer heat from a first portion of an object to a second object or a second portion of the same object, thus cooling the first portion of the object. Fluid in contact with and around the first portion of the object is thereby cooled. There are other types of cooling that could be used with these teachings and the provided cooler types are merely examples.

The mechanical coupling between the conduit 134 and the fluid cooler 228 can retain a first end of the conduit 134 about a port of the fluid cooler 228. The mechanical coupling can include a form fit, a compression ring, or other mechanical coupling.

The control circuitry 142 can provide a control signal on communication medium 220 to the fluid cooler 228. The control signal can cause the fluid cooler 228 to adjust a temperature to which the fluid cooler 228 cools the fluid. The control signal can cause an increase in the temperature set point of the fluid cooler 228 (or turn off the cooling of the fluid cooler 228) if the temperature data on communication medium 140 indicates a temperature satisfied the third or fourth criterion. The control signal can cause a decrease in the temperature set point of the fluid cooler 228 if the temperature data on communication medium 140 indicates the temperature satisfied the first or second criterion.

The control circuitry 142 can provide display temperature data on communication medium 222. The display temperature data can be provided to a display device 224. The display temperature data can include a temperature measured or computed at the anatomic site 114, such as using the temperature sensor 138. The display temperature data can include other data associated with the temperature at the anatomic site 114, such as the temperature set point of the fluid cooler 228, the power or other energy delivery parameter of the energy control system, or the like.

The display device 224 can include a touchscreen, light emitting diode screen, liquid crystal display screen, or other type of display. The display device 224 can provide the display temperature data on a user interface 226. The user interface 226 provides a user with a real time (or near real time) view of the display temperature data. The user interface 226 can include an application programming interface (API) that allows a user to provide system control parameters that govern operation of the control circuitry 142. The system control parameters can include a temperature threshold (a high temperature threshold, a low temperature threshold, or both), a maximum amount of power to be provided by the energy delivery device 118 via the energy control system 116, an electrical or optical energy parameter of energy to be provided by the energy control system 116, a temperature set point of the fluid cooler 228, a combination thereof, or the like. The control parameters provided through the user interface 226 can be implemented by the control circuitry 142.

The user interface 226 can provide an alert to the user if the temperature data, pressure data, flow rate data, or a combination thereof indicates that the temperature, pressure, or a combination thereof at the anatomic site 114 is getting near, equals, or has satisfied a criterion. The alert can be visual, using the interface 226. For example, a visual alert can include a picture, video, text, a combination thereof, or the like. In one embodiment, the user interface 226 continually displays a temperature reading/estimate in some neutral color (e.g., white) when the measured/computed temperature at the anatomic site 114 is below the predetermined threshold. Once the measured/computed temperature at the anatomic site 114 exceeds the threshold, then the color of the numbers may change to red and/or bolded. Additionally, or alternatively, an audio, haptic feedback, or other alert can be used to indicate that the temperature data, pressure data, flow rate data, or a combination thereof indicates the temperature, pressure, or a combination thereof is getting near, equals, or otherwise satisfies specified criterion. The display of the temperature data, pressure data, flow rate data, or a combination thereof can allow the user to make a manual adjustment to the fluid cooler 228 (e.g., to adjust the temperature of the fluid), the fluid reservoir 102 (e.g., to adjust the flow rate of the fluid), or energy control system 116 (e.g., to adjust a temperature change rate of the energy provided to the anatomic site 114) that better retains the temperature at the anatomic site 114 within determined limits. The user interface 226 can provide a view, audio, haptic feedback, or the like, of a suggested adjustment that, if approved by the user through the user interface 226, is automatically implemented by the control circuitry 142. In some embodiments, the control circuitry 142 can perform a suggested adjustment after a specified amount of time has elapsed or automatically without delay. A user can define acceptable ranges within which the system 200 (or other system) is allowed to make automatic adjustments to the energy control system 116, the fluid reservoir 102, or the fluid cooler 228. For example, a user can allow the system 200 to automatically adjust the energy control system 116 to deliver up to 20 W of energy, and 20 Hz of pulse frequency, and 150 mm water of pressure, such as to try to keep the temperature about the anatomic site 114 below 42° C. These approved system operation ranges may be configured by the user so the user can reduce how frequently he/she needs to respond to system alerts or suggestions while treating patients.

The system 200 provides for increased cooling control, such as when the energy delivery device 118 provides energy at a rate that causes a temperature change that is not manageable by un-cooled fluid. The system 200 can allow a user to operate the energy delivery device 118 at the anatomic site 114 for a longer duration than would be possible without actively cooling the fluid. The use of room temperature fluid, or other fluid whose temperature is not active managed provides for less precise control of the temperature at the anatomic site 114. Using the fluid cooler 228 allows the user to operate the energy delivery device 118 for longer periods of time without raising the temperature above the de-naturing temperature than what is accommodated by room temperature fluid. This allows the user to provide therapy more continuously without having to pause and wait for the temperature at and around the anatomic site 114 to reduce to a temperature that does not jeopardize tissue around the anatomic site 114.

FIG. 3 illustrates, by way of example, a block diagram of another system 300 for management of fluid temperature of the fluid provided to the anatomic site 114. The system 300 includes some components from the system 100A, 100B, or 200 including the fluid reservoir 102, the conduit 134, the scope 120, the energy control system 116, the energy delivery device 118, the temperature sensor 138, the control circuitry 142, the display device 224, the fluid cooler 228, and the communication mediums 136, 140, 146, 220, 222. The system 300 includes additional components including a suction device 330, a flow sensor 336, a temperature sensor 338, a waste receptacle 334, a second conduit 332, and communication mediums 340 and 342.

The suction device 330 removes fluid and debris from anatomic site 114. The suction device 330 can generate a negative air pressure that causes fluid and debris to flow through the conduit 332 to the waste receptacle 334. The suction device 330 can be formed integral with the scope 120, be physically separate from the scope 120, attached to the scope 120, detachably coupled to the scope 120, or the like. The suction device 330 can help remove warm fluid and debris from the anatomic site 114 and thus helps in maintaining the temperature about the anatomic site 114.

The conduit 332 can extend between the suction device 330 and the waste receptable. The conduit 332 can transport fluid and debris from the anatomic site 114 to the waste receptacle 334. The conduit 332 and the conduit 134 can be different portions of the same conduit or can be discrete conduits.

The temperature sensor 338 can be on, or at least partially in, the suction device 330 or conduit 332. The temperature sensor 338 can provide temperature data to the control circuitry 142 through communication medium 340. The control circuitry 142 can determine a temperature associated with the anatomic site 114 based on the temperature data. The temperature of fluid removed by the suction device 330 can be lower than the temperature at the anatomic site 114. The temperature from the temperature sensor 338 can be adjusted (e.g., by a constant, proportional to the temperature, or based on the temperature) to account for the cooling as the fluid is removed from the anatomic site 114. The difference between the measured temperature data and the temperature at the anatomic site 114 may be empirically determined and/or theoretically computed prior to the therapeutic procedure.

The flow sensor 336 can be on, or at least partially in, the suction device 330 or conduit 332. The flow sensor 336 can provide flow data to the control circuitry 142 through communication medium 342. The flow data can indicate how fast fluid and debris is being removed from the anatomic site 114. The user or control circuitry 142 can adjust a setting of the suction device 330, such as a flow rate of the suction device 330. The control circuitry 142 can change the flow rate of the suction device 330 by issuing a control signal on the communication medium 344. The control circuitry 142 can determine a temperature associated with the anatomic site based on the temperature data from the temperature sensor 338, the temperature data from the temperature sensor 138, the flow data from the flow sensor 336, or a combination thereof. The control circuitry 142 can increase the flow rate of the fluid in the conduit 134 and/or the suction device 330, such as to alter the temperature at the anatomic site 114. The control circuitry 142 can alter the temperature set point of the fluid cooler 228 based on the temperature data from the temperature sensor 338, the flow data from the flow sensor 336, the temperature data from the temperature sensor 138, or a combination thereof

The user interface 226 can provide a view of the flow data, temperature data, or other data provided to the control circuitry 142. The user interface 226 can provide a user with a view of the current setting of the suction device 330, such as the flow rate or another parameter associated with the flow rate, such as a pump rate of a pump associated with the suction device 330, or the like.

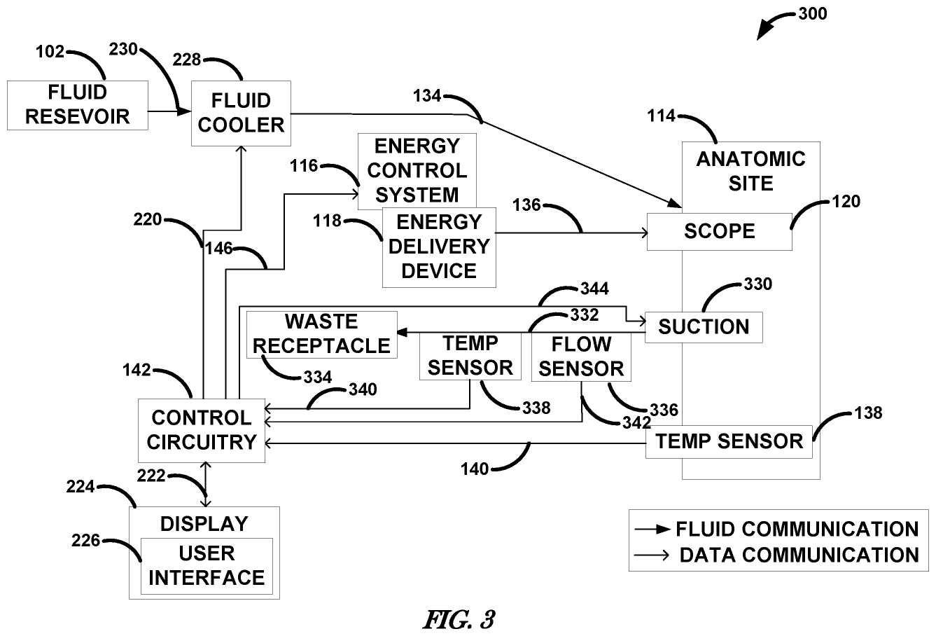

FIG. 4 illustrates, by way of example, a block diagram of a system 400 for management of fluid temperature of a mixed fluid provided to or pressure at the anatomic site 114. The system 400 includes some components from the systems 100A, 100B and 200 and includes some additional components. The system 400 can include the suction device 330, temperature sensor 338, flow sensor 336, the conduit 332, one or more communication mediums 344, 342, 340, 222, display device 224, or a combination thereof. The additional components in FIG. 4 include fluid reservoir 440, conduit 442, actuated valve 444, temperature sensor 446, conduit 448, pump 450, flow sensor 452, and communication mediums 456, 458, 460, 462.

The fluid reservoir 440 can be a same source or a different source as the fluid reservoir 102. The fluid in the fluid reservoir 440 can be saline or another fluid. The fluid from the fluid reservoir 440 can travel through conduit 442 to the actuated valve 444. Temperature of the fluid from the fluid reservoir 440 is sometimes called a “direct fluid” because it does not go through a heater or cooler before being delivered to the anatomic site 114.

The actuated valve 444 can receive both the fluid from the fluid reservoir 440 and cooled fluid from the fluid cooler 228. The cooled fluid can be provided a conduit 466 coupled between the fluid cooler 228 and the actuated valve 444. The actuated valve 444 can cause the fluid from the fluid reservoir 440 and the cooled fluid to mix. An effective size of an output orifice can be adjusted by a control signal of the control circuitry 142 on communication medium 456. For example, the control signal can cause a motor coupled to the output orifice to increase or decrease an opening provided by the orifice. The control circuitry 142 can thus titrate how much fluid is provided at the output of the actuated valve 444 and is ultimately provided to the anatomic site 114. The actuated valve includes a valve actuator that uses a power source coupled to a motor of the actuated valve 444 that is mechanically coupled to operate the valve. The power source can be electrical, pneumatic, or hydraulic. The actuated valve 444 can be rotary or linear.

The control circuitry 142 can increase the amount of fluid provided to the anatomic site 114 by opening one or more orifices of the actuated valve 444. The control circuitry 142 can decrease the amount of fluid provided to the anatomic site 114 by closing one or more orifices of the actuated valve 444. The control circuitry 142 can modulate an opening in an orifice in the actuated valve 444 by issuing a control signal on the communication medium 456.

By adjusting the opening size of one or more orifices of the actuated valve 444, the control circuitry 142 can adjust the temperature of the fluid in the conduit 134 and thus alter the temperature of the fluid provided to the anatomic site 114. For example, there may be two separate orifices (or valves), respectively associated with the direct fluid and cooled fluid. By adjusting the state (e.g., size of opening or the opening degree) of each orifice (or valve), one can control the temperature of the mixed fluid in the conduit 134. The temperature of the mixed fluid can be determined based on the temperature associated with the anatomic site 114, the length of the conduit 134 between the valve 444 and the anatomic site 114, or a combination thereof. The temperature of the anatomic site 114 can be compensated to account for heating or cooling that occurs along the length of the conduit 134, by energy delivered by the energy delivery device 118, air flow provided by the suction device 330, a combination thereof, or the like. The temperature of the mixed fluid can be regulated to ensure that the temperature at the anatomic site 114 remains within a user-specified (or default) range of acceptable temperatures.

The temperature sensor 446 can be on, or at least partially in, the actuated valve 444. The temperature sensor 446 can provide temperature data indicating a temperature of the mixed fluid in the actuated valve 444. The temperature data can be provided to the control circuitry 142 using the communication medium 458.

The conduit 448 can provide mixed fluid from the actuated valve 444 to the pump 450. The pump 450 can include a peristaltic pump or a similar fluid pump. The pump 450 can include an adjustable pump rate that affects a flow rate of the mixed fluid in the conduit 134. The pump rate of the pump 450 can be adjusted by the control circuitry 142. The control circuitry 142 can issue a control signal on communication medium 460 that adjusts a pump rate of the pump 450.

The flow sensor 452 can provide flow data to the control circuitry 142 on communication medium 462. The flow data can indicate how much fluid passes into the conduit 134 per unit time. The control circuitry 142 can increase a pump rate (and the flow rate of mixed fluid) to decrease the temperature at the anatomic site 114.

The pressure sensor 150 can provide the control circuitry 142 with pressure data over communication medium 154. The pressure data can indicate a fluid pressure near the anatomic site and/or a pressure on tissue of the anatomic site 114. The pressure sensor 150 can be integrally formed with the scope 120, detachably coupled to the scope 120, attached to the scope 120, or physically separate from the scope 120. The pressure at the anatomic site 114 can indicate how much gas is being retained from fluid evaporation, how much fluid and debris is building up in the anatomic site 114, a temperature associated with the anatomic site 114, or a combination thereof. The pressure at the anatomic site 114 can affect the efficacy of the energy delivered to the anatomic site 114. By controlling the pressure, the control circuitry 142 can help ensure that the energy delivery is effective and safe.

The user interface 226 (see FIGS. 2 and 3) can provide a view of the temperature data from the temperature sensor 446, a state of the actuated valve 444 (an amount that the valve is opened or closed), a pump rate of the pump 450, a flow rate of mixed fluid in the conduit 134, pressure data from the pressure sensor 150, or a combination thereof. The user, with the data provided on the user interface 226, can be better informed as to the state and efficacy of energy delivery.

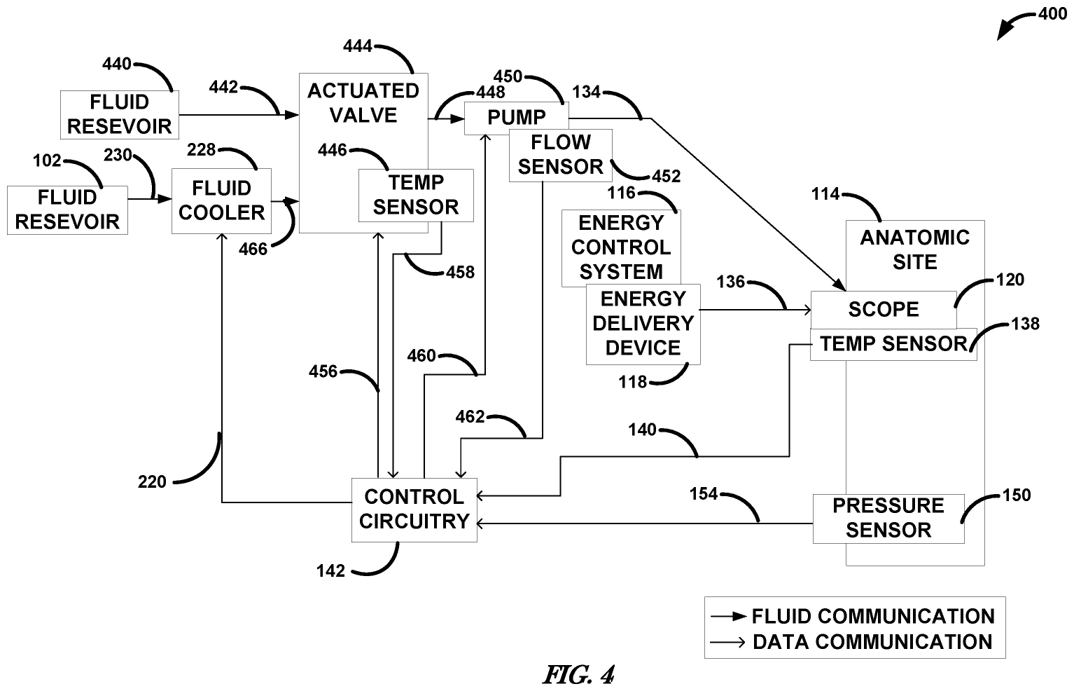

FIG. 5 illustrates, by way of example, a block diagram of a system 500 for management of fluid temperature of a mixed fluid provided to the anatomic site 114. The system 500 includes components of the systems 100A, 100B, 200, 300, and 400. The system 500 includes additional components including another fluid reservoir 550, a conduit 552, fluid heater 554, a conduit 556, a communication medium 558, a communication medium 562 and an alerting device 560. The system 500 includes fluids of three different temperatures being mixed at the actuated valve 444. The fluids include the cooled fluid from the fluid cooler 228, the direct fluid from the fluid reservoir 440, and heated fluid from the fluid heater 554. While fluids of three different temperatures are illustrated, fluids of only two different temperatures or fluids of more than three temperatures can be used.

The fluid reservoir 550 can be a same or different fluid source as the fluid reservoir 440 or the fluid reservoir 102. The fluid heater 554 can receive the fluid from the fluid reservoir 550 by the conduit 552. The fluid heater 554 can include an electrical, electromagnetic, ceramic, or other heater. The fluid heater 554 can increase the temperature of the fluid from the fluid reservoir 550. The heated fluid can be provided to the actuated valve 444 by conduit 556.

The control circuitry 142 can adjust a temperature set point of the fluid heater 554. The control circuitry 142 can provide a control signal on communication medium 558 that causes a change in the temperature set point of the fluid heater 554. The control circuitry 142 can increase the temperature setpoint of the fluid heater 554 to increase a temperature of the fluid provided through the conduit 134 to the anatomic site 114. The control circuitry 142 can decrease the temperature setpoint of the fluid heater 554 to decrease a temperature of the fluid provided through the conduit 134 to the anatomic site 114.

By adjusting the opening size of one or more orifices of the actuated valve 444, the control circuitry 142 can adjust the temperature of the fluid in the conduit 134 and thus alter the temperature of the fluid provided to the anatomic site 114. For example, there may be three separate orifices (or valves), respectively associated with the heated fluid, direct fluid, and cooled fluid. By adjusting the state (e.g., size of opening or the opening degree) of each orifice (or valve), one can control the temperature of the mixed fluid in the conduit 134. The temperature of the mixed fluid can be determined based on the temperature associated with the anatomic site 114, the length of the conduit 134 between the valve 444 and the anatomic site 114, or a combination thereof. The temperature of the anatomic site 114 can be compensated to account for heating or cooling that occurs along the length of the conduit 134, by energy delivered by the energy delivery device 118, air flow provided by the suction device 330, a combination thereof, or the like. The temperature of the mixed fluid can be regulated to ensure that the temperature at the anatomic site 114 remains within a user-specified (or default) range of acceptable temperatures.

The user interface 226 can provide data indicating the temperature set point of the fluid heater 554 or another temperature set point. A user can adjust the temperature set point of the fluid heater 554 or another temperature set point through the user interface 226.

By keeping track of the irrigation inflow and out flow volumes, the temperature of irrigation inflow and outflow, and the energy settings of the energy control system 116 (e.g., Joules per pulse, watts, or the like) the control circuitry 142 can monitor the amount of energy having gone into and out of the anatomic site 114. Equation (1) demonstrates this relation

$$T = {T}_{inflow} + 0.239 Δ E / Δ V \qquad ( 1 )$$

ΔE is energy accumulative difference (in Joules or the like) at a moment in time, ΔV is the difference in the amount of irrigation fluid going into and out of the surgical space at the same moment in time (e.g., in cubic centimeters), T_{inflow} is the temperature of the fluid flowing into the anatomic site 114 and T is the average temperature of the irrigation fluid at the moment in time inside the anatomic site 114. More details regarding how to determine temperature at the anatomic site are provided in United States Patent Publication Number 2018/0055568 titled “Automatic Irrigation-Coordinated Lithotripsy”, and filed on Aug. 25, 2018, which is incorporated herein by reference in its entirety.

The control circuitry 142 can use Equation (1) to estimate the temperature of the anatomic site 114 by monitoring, over the same time duration, the inflow and outflow irrigation volumes, the energy added to the anatomic site 114 by the energy delivery device 118, and the temperature of the inflow fluid, (e.g., saline).

The communication medium 562 can provide alert data to the alerting device 560. The alerting device 560 can include a display, speaker, motor, or the like configured to provide an indication that a temperature threshold has been violated. The display 224 can additionally or alternatively provide the alert data. The speaker can provide audio that indicates the threshold has been violated, which threshold has been violated, or a combination thereof. The motor can provide vibrations (haptic feedback) that indicates the threshold has been violated, which threshold have been violated, or a combination thereof. The display can provide a visual alert indicating the threshold has been violated, which threshold has been violated, or a combination thereof.

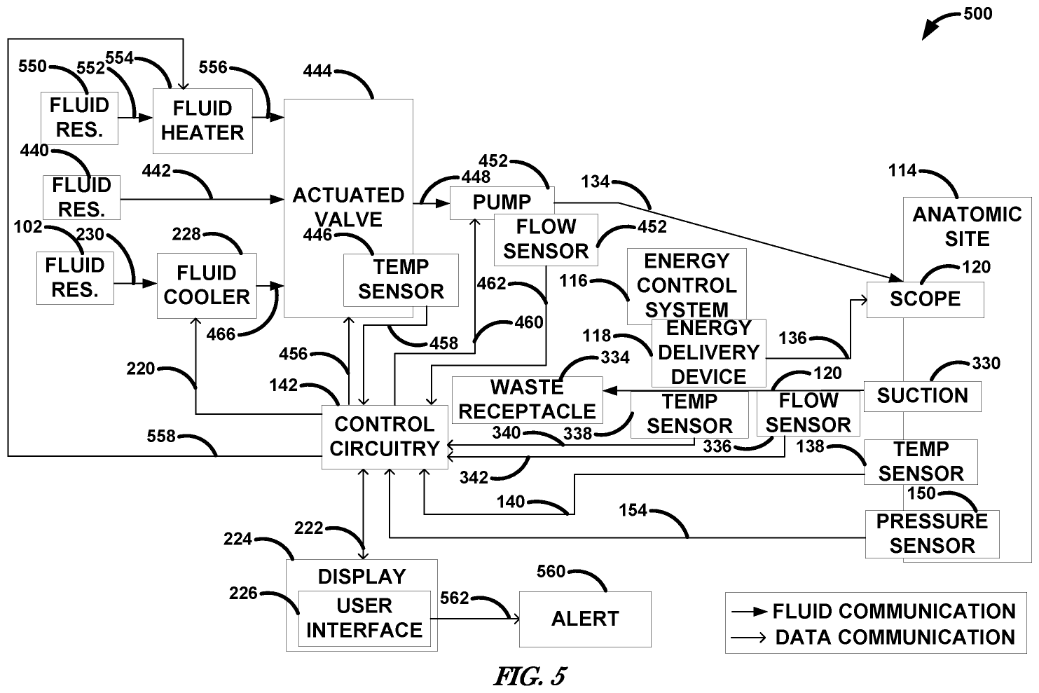

FIG. 6 illustrates, by way of example, a diagram of an embodiment of a method 600 for temperature management of an anatomic site. The method 600 can include operations performed by one or more of the components of one or more of the systems 100A, 100B, 200, 300, 400, 500, or a combination thereof. The method 600 as illustrated includes providing an optical or electrical energy to an anatomic site, at operation 660; providing, via a first conduit, fluid to the anatomic site, at operation 662; receiving, at control circuitry, a first temperature associated with the anatomic site, at operation 664; and providing, by the control circuitry and based on the first temperature, a control signal that titrates at least one of a second temperature or a flow parameter of the fluid to the anatomic site to manage a temperature of the anatomic site toward a desired target temperature, at operation 666.

The method 600 can further include removing, by a suction device, fluid from the anatomic site resulting in removed fluid. The method 600 can further include transferring, by a second conduit in fluid communication with the suction device, the removed fluid to a waste fluid dispenser. The first temperature can be a temperature of the removed fluid or of the fluid at the anatomic site.

The method 600 can further include cooling, by a fluid cooler, a first portion of the fluid resulting in cooled fluid. The method 600 can further include providing, by a second conduit, the cooled fluid. The method 600 can further include, adjusting, by the control circuitry and based on the first temperature, a second temperature to which the fluid cooler cools the cooled fluid.

The method 600 can further include heating, by a fluid heater, a second portion of the fluid resulting in heated fluid. The method 600 can further include providing, by a third conduit, the heated fluid. The method 600 can further include, adjusting, by the control circuitry and based on the first temperature, a third temperature to which the fluid heater heats the cooled fluid.

The method 600 can further include providing, by a display electrically coupled to the control circuitry, a view of the first temperature, a temperature set point of the fluid heater, a temperature set point of the fluid cooler, a flow rate of fluid to the anatomic site, a flow rate of fluid away from the anatomic site, a temperature of mixed water, a pressure at the anatomic site, a temperature of fluid flowing away from the anatomic site, a pump rate of a pump, or a combination thereof.

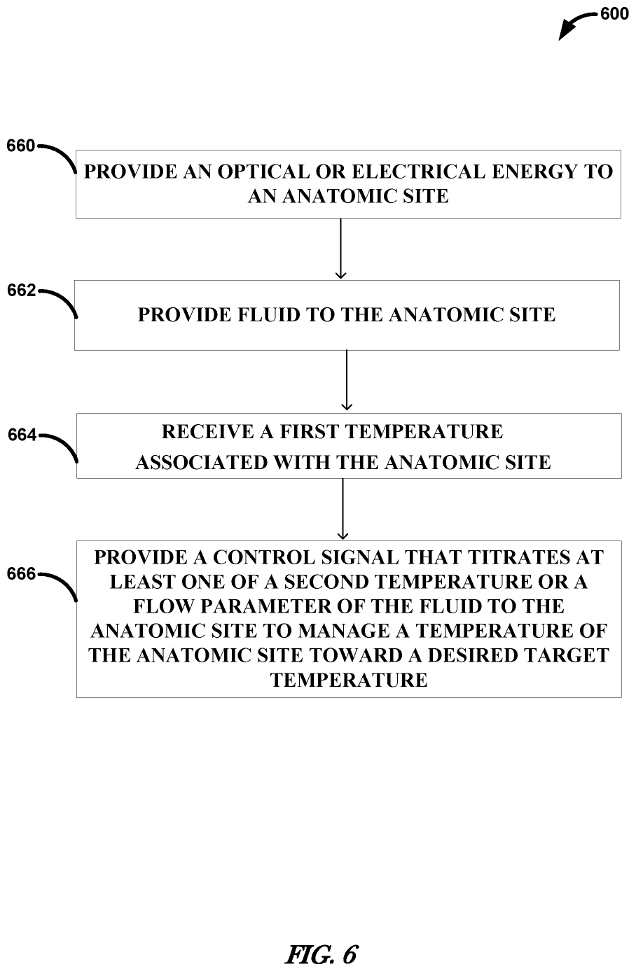

FIG. 7 illustrates, by way of example, a flow diagram of a method 700 for determining a temperature, pressure, or a combination thereof, at the anatomic site 114. The method 700 can be performed, for example, by the control circuitry 142. The method 700 as illustrated includes receiving energy data 770, temperature data 772, pressure data 774, flow rate data 776, or a combination thereof. The method 700 as illustrated further includes determining the temperature or pressure associated with the anatomic site 114 at operation 778.

The energy data 770 regards one or more settings of the energy control system 116. The settings define the operating parameters of the energy control system 116. The operating parameters can include an amplitude, frequency, voltage, current, phase, power, a combination thereof, or the like. The energy data 770 can be provided by the energy control system 116 over communication medium 146 or known by the control circuitry 142 (because the control circuitry 142 can set the operating parameters).

The temperature data 772 can include data from any of the temperature sensors of any of the systems 100A, 100B, 200, 300, 400, or 500. The temperature data 772 can be associated with the fluid into the anatomic site 114, the fluid coming out of the anatomic site 114, or an ambient temperature of the room in which the system 100A, 100B, 200, 300, 400, 500 is situated. The temperature data can include a temperature set point of the fluid cooler 228, fluid heater 554, or a combination thereof.

The pressure data 774 can indicate a measure pressure at the anatomic site 114. The pressure data 774 can indicate a fluid pressure, a gas pressure, or an overall pressure at the anatomic site 114. The pressure data 774 can be from the pressure sensor 150, or another pressure sensor.

The flow rate data 776 can indicate a rate at which fluid is flowing in a particular confined region. The flow rate data 776 can be from any of the flow rate sensors 148, 336, 452, another flow rate sensor, the pump 450, the suction device 330, or a combination thereof. The flow rate data 776 can indicate a flow rate or a pump rate. The flow rate data 776 can indicate a volume per unit time that is moved by the pump 450.

The operation 778 can include estimating the temperature, pressure, or both at the anatomic site 114. Pressure can be determined based the pressure data 774, the temperature data 772, the flow rate data 776, the energy data 770, or a combination thereof. An increase in temperature generally means there is an increase in pressure. The higher the delta between the flow rate into and out of the anatomic site 114, the higher the pressure. The higher the amount of energy being provided to the anatomic site 114, the higher the temperature is at the anatomic site and thus the higher the pressure. The control circuitry 142 can weigh all, or just a subset, of these factors in determining the pressure at operation 778.

The temperature can be determined based the pressure data 774, the temperature data 772, the flow rate data 776, the energy data 770, or a combination thereof. An increase in pressure generally means there is an increase in temperature. The higher the flow rate of fluid into the anatomic site 114, the closer the temperature of the anatomic site 114 matches the temperature of the fluid into the anatomic site 114. The higher the amount of energy being provided to the anatomic site 114, the higher the temperature is at the anatomic site 114. The control circuitry 142 can weigh all, or just a subset, of these factors in determining the temperature at operation 778.

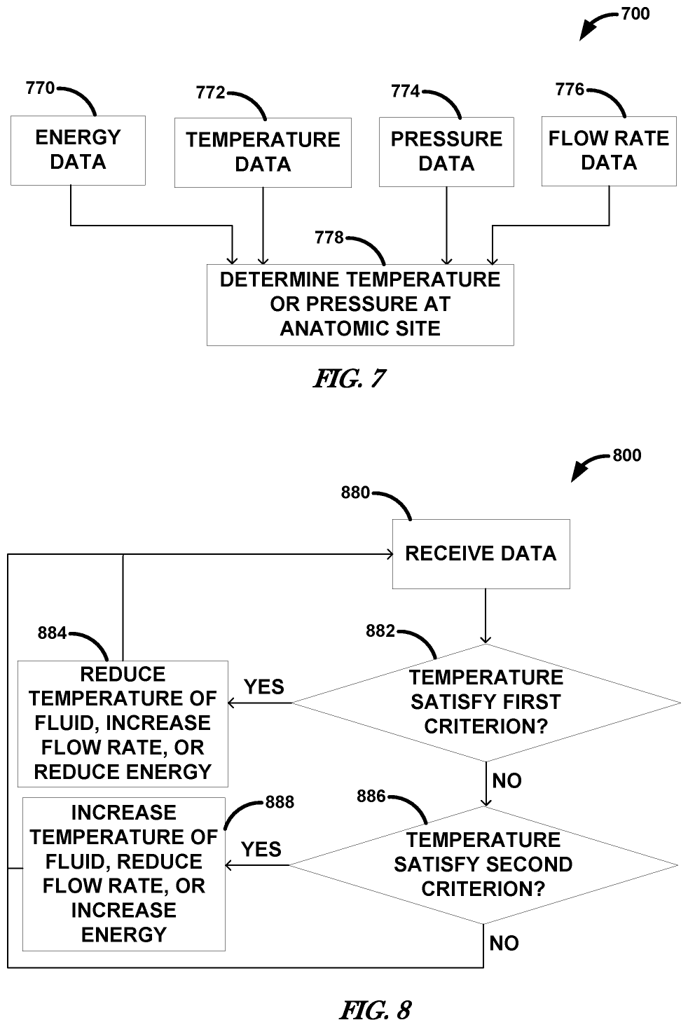

FIG. 8 illustrates, by way of example, a diagram of a method 800 for adjusting one or more components of the system 100A, 100B, 200, 300, 400, or 500 to regulate temperature at the anatomic site 114. The method 800 can be performed, at least in part, by the control circuitry 142. The method 800 as illustrated includes receiving data at operation 880. The received data can include any of the pressure data 774, the temperature data 772, the flow rate data 776, the energy data 770, or a combination thereof (see FIG. 7). The operation 880 can include determining the temperature associated with the anatomic site (such as by performing operation 778 of FIG. 7). At operation 882, the temperature can be compared to first criterion. If the temperature satisfies the first criterion (indicating the temperature is too high or trending towards being too high) an operation 884 can be performed. If the temperature does not satisfy the first criterion operation 886 can be performed.

The first criterion can include a temperature being greater than a specified threshold temperature, a rate of change of the temperature being, for example, positive and greater than another specified threshold, a combination thereof, or the like. The operation 884 can include (e.g., responsive to determining the temperature satisfies the first criterion) (i) reducing a temperature of the fluid into the anatomic site 114, such as by reducing a temperature set point of the fluid heater 554, reducing a temperature set point of the fluid cooler 228, decreasing an opening in an orifice of the actuated valve that provides heated fluid, or increasing an opening in an orifice of the actuated valve that provides cooled fluid, direct fluid, or a combination thereof, (ii) increasing an amount of fluid provided to the anatomic site 114, such as by increasing a pump rate of the pump 450, increasing an opening in an orifice of the actuated valve that provides cooled fluid, direct fluid, or a combination thereof, (iii) increasing an amount of fluid and debris removed from the anatomic site 114, such as increasing a pump rate of the suction device 330, or (iv) reducing an amount of energy provided by the energy delivery device 118, a combination thereof, or the like.

At operation 886, the temperature can be compared to second criterion. If the temperature satisfies the second criterion (indicating the temperature is too low or trending towards being too low) an operation 888 can be performed. If the temperature does not satisfy the second criterion operation 880 can be performed.

The second criterion can include a temperature being less than a specified threshold temperature, a rate of change of the temperature being, for example, negative with a magnitude greater than another specified threshold, a combination thereof, or the like. The operation 888 can include (e.g., responsive to determining the temperature satisfies the second criterion) (i) increasing a temperature of the fluid into the anatomic site 114, such as by increasing a temperature set point of the fluid heater 554, increasing a temperature set point of the fluid cooler 228, increasing an opening in an orifice of the actuated valve that provides heated fluid, or decreasing an opening in an orifice of the actuated valve that provides cooled fluid, direct fluid, or a combination thereof, (ii) decreasing an amount of fluid provided to the anatomic site 114, such as by decreasing a pump rate of the pump 450, decreasing an opening in an orifice of the actuated valve that provides cooled fluid, direct fluid, or a combination thereof, (iii) increasing an amount of fluid and debris removed from the anatomic site 114, such as increasing a pump rate of the suction device 330, or (iv) increasing an amount of energy provided by the energy delivery device 118, a combination thereof, or the like.

Note that removing fluid and debris from the anatomic site 114 can serve to increase the temperature at the anatomic site 114, such as when the temperature of the fluid at the anatomic site 114 is below ambient temperature and can serve to decrease the temperature at the anatomic site 114, such as when the temperature of the fluid at the anatomic site 114 is above ambient temperature.

There are many relationships that can be derived from the measurements of flow rate, temperature, time, pressure, or a combination thereof. One such relationship can include determining a rate of temperature change (a temperature change per unit time) which can then be projected forward in time to estimate how much longer it will take to arrive at a user defined temperature threshold. For example, there could be a countdown displayed on the user interface 226 that displays how much time is left before the temperature at the anatomic site 114 is predicted to reach one of the thresholds. The user interface 226 can provide a view of the measurements, predictions, or the like to users such as to provide them with information about rates of change and time to exceed thresholds so the user can make the appropriate decisions about what is best for the patient.

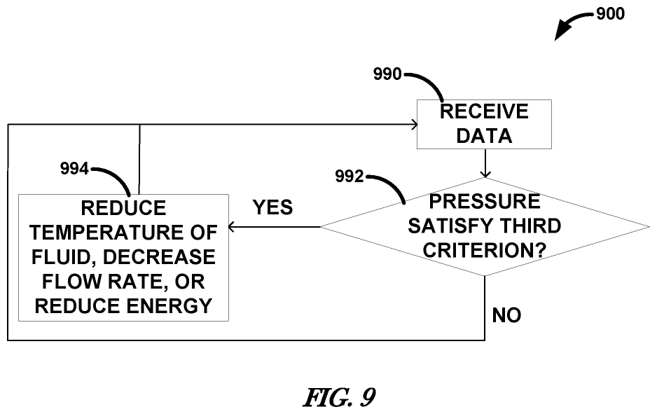

FIG. 9 illustrates, by way of example, a diagram of a method 900 for adjusting one or more components of the system 100A, 100B, 200, 300, 400, or 500 to regulate pressure at the anatomic site 114. The method 900 can be performed, at least in part, by the control circuitry 142. The method 900 as illustrated includes receiving data at operation 990. The received data can include any of the pressure data 774, the temperature data 772, the flow rate data 776, the energy data 770, or a combination thereof (see FIG. 7). The operation 990 can include determining the pressure associated with the anatomic site (such as by performing operation 778 of FIG. 7). The method 900 as illustrated includes determining whether the pressure satisfies a third criterion at operation 992. If the pressure satisfies the third criterion (indicating the pressure is too high or trending towards being too high) an operation 994 can be performed. If the pressure does not satisfy the third criterion operation 990 can be performed.

The third criterion can include the pressure being greater than a specified pressure threshold, a rate of change of the pressure being, for example, positive and greater than another specified pressure threshold, a combination thereof, or the like. The operation 994 can include (e.g., responsive to determining the pressure satisfies the third criterion) (i) decreasing a temperature of the fluid into the anatomic site 114, such as by decreasing a temperature set point of the fluid heater 554, decreasing a temperature set point of the fluid cooler 228, decreasing an opening in an orifice of the actuated valve 444 that provides heated fluid, or increasing an opening in an orifice of the actuated valve 444 that provides cooled fluid, direct fluid, or a combination thereof, (ii) decreasing an amount of fluid provided to the anatomic site 114, such as by decreasing a pump rate of the pump 450, decreasing an opening in an orifice of the actuated valve 444 that provides cooled fluid, heated fluid, direct fluid, or a combination thereof, (iii) increasing an amount of fluid and debris removed from the anatomic site 114, such as increasing a pump rate of the suction device 330, or (iv) decreasing an amount of energy provided by the energy delivery device 118, a combination thereof, or the like.

One or more of the systems 100A, 100B, 200, 300, 400, 500, such as while implementing one or more the methods 600, 700, 800, or 900 can maintain temperatures at the anatomic site 114 close to a specified temperature (e.g., 37 deg. C/normal body temp), which in turn allows physicians to use higher power therapy systems that result in more efficient/faster procedure times. With the extra heat being generated by higher power energy therapeutic systems, teachings provide refrigerated (or otherwise cooled) irrigation fluid. The teachings can use a feedback-driven system to control fluid temperature at the site.

With cooled irrigation fluid, the temperature at the anatomic/procedure site can be controlled by adding the appropriate amount of cooled fluid if/when needed or to add heat when the temp gets too cool. Such a system can include a valve, and room temp saline, cooled saline, and/or heated saline can be one or more inputs to the valve to enable the irrigation fluid temperature to be accurately controlled. Feedback temperatures, such as from the anatomic site or excess fluid, can provide temperature and flow information for the Logic to make decisions as to how to control the valve and irrigation pump.

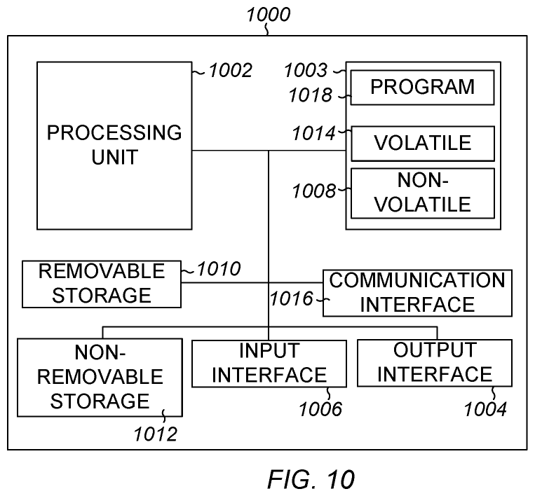

FIG. 10 illustrates, by way of example, a block diagram of an embodiment of a machine 1000 (e.g., a computer system) to implement one or more embodiments. The machine 1000 can implement a method for temperature management of an anatomic site, such as the method 600, 700, 800, 900, a portion thereof, or a combination of the whole or portion. The control circuitry 142, energy control system 116, energy delivery device 118, scope 120, temperature sensor 138, fluid cooler 228, display 224, flow sensor 336, temperature sensor 338, actuated valve 444, pump 450, flow sensor 452, temperature sensor 446, pressure sensor 150, fluid heater 554, or a combination thereof can include one or more components of the machine 1000.

One example machine 1000 (in the form of a computer), can include a processing unit 1002, memory 1003, removable storage 1010, and non-removable storage 1012. Although the example computing device is illustrated and described as machine 1000, the computing device can be in different forms in different embodiments. For example, the computing device can instead be a smartphone, a tablet, smartwatch, or other computing device including the same or similar elements as illustrated and described regarding FIG. 10. Devices such as smartphones, tablets, and smartwatches are generally collectively referred to as mobile devices. Further, although the various data storage elements are illustrated as part of the machine 1000, the storage can also or alternatively include cloud-based storage accessible via a network, such as the Internet.

Memory 1003 can include volatile memory 1014 and non-volatile memory 1008. The machine 1000 can include—or have access to a computing environment that includes—a variety of computer-readable media, such as volatile memory 1014 and non-volatile memory 1008, removable storage 1010 and non-removable storage 1012. Computer storage includes random access memory (RAM), read only memory (ROM), erasable programmable read-only memory (EPROM) & electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage or other magnetic storage devices capable of storing computer-readable instructions for execution to perform functions described herein.

The machine 1000 can include or have access to a computing environment that includes input 1006, output 1004, and a communication connection 1016. Output 1004 can include a display device, such as a touchscreen, that also can serve as an input device. The input 1006 can include one or more of a touch screen, touchpad, mouse, keyboard, camera, one or more device-specific buttons, one or more sensors integrated within or coupled via wired or wireless data connections to the machine 1000, and other input devices. The computer can operate in a networked environment using a communication connection to connect to one or more remote computers, such as database servers, including cloud-based servers and storage. The remote computer can include a personal computer (PC), server, router, network PC, a peer device or other common network node, or the like. The communication connection can include a Local Area Network (LAN), a Wide Area Network (WAN), cellular, Institute of Electrical and Electronics Engineers (IEEE) 802.11 (Wi-Fi), Bluetooth, or other networks.

Computer-readable instructions stored on a computer-readable storage device are executable by the processing unit 1002 (sometimes called processing circuitry) of the machine 1000. A hard drive, CD-ROM, and RAM are some examples of articles including a non-transitory computer-readable medium such as a storage device. For example, a computer program 1018 can be used to cause processing unit 1002 to perform one or more methods or algorithms described herein.

Additional Notes and Examples

Example 1 includes a therapy delivery system comprising a scope configured to provide a view of an anatomic site, an energy delivery device configured to deliver therapy energy to the anatomic site, a first irrigation conduit configured to transfer fluid to the anatomic site, a first temperature sensor situated to provide first temperature data associated with the anatomic site, and control circuitry electrically coupled to receive the first temperature data, the control circuitry being configured to adjust, based at least in part on the first temperature data, at least one of (i) a first temperature of the fluid, (ii) a flow parameter of the fluid, or (iii) a setting of the energy delivery device to manage a second temperature of the anatomic site.

In Example 2, Example 1 can further include a suction device configured to remove fluid from the anatomic site resulting in removed fluid, and a second irrigation conduit in fluid communication with the suction device to receive the removed fluid and configured to transfer the removed fluid away from the anatomic site, wherein the first temperature data is a third temperature of the removed fluid.

In Example 3, at least one of Examples 1-2 can further include, wherein the first temperature data includes the second temperature.

In Example 4, at least one of Examples 1-3 can further include a fluid cooler in fluid communication with the fluid, the fluid cooler configured to receive and cool a first portion of the fluid resulting in cooled fluid, wherein the control circuitry adjusts a fourth temperature to which the fluid cooler cools the cooled fluid based on the first temperature data.

In Example 5, Example 4 can further include a third irrigation conduit in fluid communication with the fluid, the third irrigation conduit configured to receive a second portion of the fluid, wherein the first irrigation conduit is configured to receive a mixture of both the second portion of the fluid and the cooled fluid.

In Example 6, at least one of Examples 4-5 can further include a fluid heater in fluid communication with the fluid, the fluid heater situated to receive and heat a third portion of the fluid resulting in heated fluid, wherein the first irrigation conduit is configured to receive a mixture of both the heated and cooled fluid.

In Example 7, Example 6 can further include at least one actuated valve in fluid communication with the first irrigation conduit and electrically coupled to the control circuitry, the at least one actuated valve situated between the fluid cooler and the first irrigation conduit and/or situated between the fluid heater and the first irrigation conduit, wherein the control circuitry is configured to alter a physical state of the actuated valve based on the first temperature data.

In Example 8, Example 7 can further include a second temperature sensor situated to determine a fifth temperature of fluid out of the actuated valve, wherein the control circuitry is further configured to adjust respective temperature settings of the fluid heater and the fluid cooler based on the fifth temperature.

In Example 9, at least one of Examples 1-8 can further include a pump in fluid communication with the first irrigation conduit, the pump electrically coupled to the control circuitry, wherein the control circuitry is configured to adjust a pump rate of the pump based on the first temperature data.

In Example 10, Example 9 can further include a pressure sensor electrically coupled to the control circuitry and situated to generate pressure data representing pressure about the anatomic site, wherein the control circuitry is further configured to adjust a pump rate of the pump based on the pressure data.

In Example 11, at least one of Examples 9-10 can further include a flow sensor situated to determine a flow rate of fluid from the pump, the flow sensor electrically coupled to the control circuitry, wherein the control circuitry is further configured to adjust the rate of the pump based on the flow rate.

In Example 12, at least one of Examples 1-11 can further include, wherein the control circuitry is configured to adjust, based on the first temperature data, a setting of the energy delivery device to manage a temperature of the anatomic site.

In Example 13, at least one of Examples 1-12 can further include a display device electrically coupled to the control circuitry, the display device configured to provide a user a view of the first temperature.

In Example 14, Example 13 can further include an alerting device configured to generate an audio, visual, or haptic feedback indicating that the first temperature has exceeded or is about to exceed a threshold temperature.

In Example 15, at least one of Examples 13-14 can further include, wherein the display provides a user interface that is configured to receive data indicating a first temperature set point above which to maintain the anatomic site and a second temperature set point below which to maintain the anatomic site and the control circuitry automatically operates to manage the temperature of the anatomic site between first and second temperature set points.

Example 16 includes a method comprising providing an optical or electrical energy to an anatomic site, providing, via a first irrigation conduit, fluid to the anatomic site, receiving, at control circuitry, a first temperature associated with the anatomic site, and providing, by the control circuitry and based on the first temperature, a control signal that titrates at least one of a second temperature or a flow parameter of the fluid to the anatomic site to manage a temperature of the anatomic site toward a desired target temperature.

In Example 17, Example 16 can further include removing, by a suction device, fluid from the anatomic site resulting in removed fluid, and transferring, by a second irrigation conduit in fluid communication with the suction device, the removed fluid to a waste receptacle wherein the first temperature is a temperature of the removed fluid.

In Example 18, Example 17 can further include, wherein the first temperature is of the fluid at the anatomic site.

In Example 19, at least one of Examples 17-18 can further include cooling, by a fluid cooler, a first portion of the fluid resulting in cooled fluid, and providing, by a second irrigation conduit, the cooled fluid, wherein the control circuitry adjusts, based on the first temperature, a second temperature to which the fluid cooler cools the cooled fluid.

In Example 20, at least one of Examples 17-19 can further include providing, by a display electrically coupled to the control circuitry, a view of the first temperature.

Example 21 includes a therapy delivery system comprising a scope configured to provide a view of an anatomic site, an energy delivery device configured to provide an electrical or optical energy to the anatomic site, at least one actuated valve, a first irrigation conduit in fluid communication with a first portion of the fluid and the at least one actuated valve, the first irrigation conduit configured to provide the first portion directly to the actuated valve resulting in direct fluid, a fluid cooler in fluid communication with a second portion of the fluid and the at least one actuated valve, the fluid cooler configured to cool a second portion of the fluid based on a temperature setting of the fluid cooler resulting in cooled fluid, a second irrigation conduit in fluid communication with a mixture of the cooled and direct fluid, a first temperature sensor situated to provide first temperature data associated with the anatomic site, and control circuitry electrically coupled to the first temperature sensor, the control circuitry configured to receive the first temperature data and provide a control signal that adjusts a state of the at least one actuated valve, the temperature setting, or a combination thereof to manage a temperature of the anatomic site.

The preceding description and the drawings sufficiently illustrate specific embodiments to enable those skilled in the art to practice them. Other embodiments can incorporate structural, logical, electrical, process, and other changes. Portions and features of some embodiments can be included in, or substituted for, those of other embodiments. Embodiments set forth in the claims encompass all available equivalents of those claims.

Throughout this specification, plural instances can implement components, operations, or structures described as a single instance. Although individual operations of one or more methods are illustrated and described as separate operations, one or more of the individual operations can be performed concurrently, and nothing requires that the operations be performed in the order illustrated. Structures and functionality presented as separate components in example configurations can be implemented as a combined structure or component. Similarly, structures and functionality presented as a single component can be implemented as separate components. These and other variations, modifications, additions, and improvements fall within the scope of the subject matter herein.

Although an overview of the inventive subject matter has been described with reference to specific example embodiments, various modifications and changes can be made to these embodiments without departing from the broader scope of embodiments of the present disclosure. Such embodiments of the inventive subject matter can be referred to herein, individually, or collectively, by the term “invention” merely for convenience and without intending to voluntarily limit the scope of this application to any single disclosure or inventive concept if more than one is, in fact, disclosed.

The embodiments illustrated herein are described in sufficient detail to enable those skilled in the art to practice the teachings disclosed. Other embodiments can be used and derived therefrom, such that structural and logical substitutions and changes can be made without departing from the scope of this disclosure. The Detailed Description, therefore, is not to be taken in a limiting sense, and the scope of various embodiments is defined only by the appended claims, along with the full range of equivalents to which such claims are entitled.

As used herein, the term “or” can be construed in either an inclusive or exclusive sense. Moreover, plural instances can be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and can fall within a scope of various embodiments of the present disclosure. In general, structures and functionality presented as separate resources in the example configurations can be implemented as a combined structure or resource. Similarly, structures and functionality presented as a single resource can be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

The foregoing description, for the purpose of explanation, has been described with reference to specific example embodiments. However, the illustrative discussions above are not intended to be exhaustive or to limit the possible example embodiments to the precise forms disclosed. Many modifications and variations are possible in view of the above teachings. The example embodiments were chosen and described in order to best explain principles involved and their practical applications, to thereby enable others skilled in the art to best utilize the various example embodiments with various modifications as are suited to the particular use contemplated.

The terminology used in the description of the example embodiments herein is for the purpose of describing particular example embodiments only and is not intended to be limiting. As used in the description of the example embodiments and the appended examples, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” can be construed to mean “when” or “upon” or “in response to determining” or “in response to detecting,” depending on the context. Similarly, the phrase “if it is determined” or “if [a stated condition or event] is detected” can be construed to mean “upon determining” or “in response to determining” or “upon detecting [the stated condition or event]” or “in response to detecting [the stated condition or event],” depending on the context.

1. A therapy delivery system comprising:
a scope configured to provide a view of an anatomic site;
an energy delivery device configured to deliver therapy energy to the anatomic site;
a first irrigation conduit configured to transfer fluid to the anatomic site;
a first temperature sensor situated to provide first temperature data associated with the anatomic site; and
control circuitry electrically coupled to receive the first temperature data, the control circuitry being configured to adjust, based at least in part on the first temperature data, at least one of (i) a first temperature of the fluid, (ii) a flow parameter of the fluid, or (iii) a setting of the energy delivery device to manage a second temperature of the anatomic site.

2. The system of claim 1, further comprising:
a suction device configured to remove fluid from the anatomic site resulting in removed fluid; and
a second irrigation conduit in fluid communication with the suction device to receive the removed fluid and configured to transfer the removed fluid away from the anatomic site;
wherein the first temperature data is a third temperature of the removed fluid.

3. The system of claim 1, wherein the first temperature data includes the second temperature.

4. The system of claim 1, further comprising:
a fluid cooler in fluid communication with the fluid, the fluid cooler configured to receive and cool a first portion of the fluid resulting in cooled fluid;
wherein the control circuitry adjusts a fourth temperature to which the fluid cooler cools the cooled fluid based on the first temperature data.

5. The system of claim 4, further comprising:
a third irrigation conduit in fluid communication with the fluid, the third irrigation conduit configured to receive a second portion of the fluid;
wherein the first irrigation conduit is configured to receive a mixture of both the second portion of the fluid and the cooled fluid.

6. The system of claim 4, further comprising:
a fluid heater in fluid communication with the fluid, the fluid heater situated to receive and heat a third portion of the fluid resulting in heated fluid;
wherein the first irrigation conduit is configured to receive a mixture of both the heated and cooled fluid.

7. The system of claim 6, further comprising:
at least one actuated valve in fluid communication with the first irrigation conduit and electrically coupled to the control circuitry, the at least one actuated valve situated between the fluid cooler and the first irrigation conduit and/or situated between the fluid heater and the first irrigation conduit;
wherein the control circuitry is configured to alter a physical state of the actuated valve based on the first temperature data.

8. The system of claim 7, further comprising:
a second temperature sensor situated to determine a fifth temperature of fluid out of the actuated valve;
wherein the control circuitry is further configured to adjust respective temperature settings of the fluid heater and the fluid cooler based on the fifth temperature.

9. The system of claim 1, further comprising:
a pump in fluid communication with the first irrigation conduit, the pump electrically coupled to the control circuitry;
wherein the control circuitry is configured to adjust a pump rate of the pump based on the first temperature data.

10. The system of claim 9, further comprising:
a pressure sensor electrically coupled to the control circuitry and situated to generate pressure data representing pressure about the anatomic site;
wherein the control circuitry is further configured to adjust a pump rate of the pump based on the pressure data.

11. The system of claim 9, further comprising:
a flow sensor situated to determine a flow rate of fluid from the pump, the flow sensor electrically coupled to the control circuitry;
wherein the control circuitry is further configured to adjust the rate of the pump based on the flow rate.

12. The system of claim 1, wherein the control circuitry is configured to adjust, based on the first temperature data, a setting of the energy delivery device to manage a temperature of the anatomic site.

13. The system of claim 1, further comprising a display device electrically coupled to the control circuitry, the display device configured to provide a user a view of the first temperature.

14. The system of claim 13, further comprising an alerting device configured to generate an audio, visual, or haptic feedback indicating that the first temperature has exceeded or is about to exceed a threshold temperature.

15. The system of claim 13, wherein the display provides a user interface that is configured to receive data indicating a first temperature set point above which to maintain the anatomic site and a second temperature set point below which to maintain the anatomic site and the control circuitry automatically operates to manage the temperature of the anatomic site between first and second temperature set points.

16. A method comprising:
providing an optical or electrical energy to an anatomic site;
providing, via a first irrigation conduit, fluid to the anatomic site;
receiving, at control circuitry, a first temperature associated with the anatomic site; and
providing, by the control circuitry and based on the first temperature, a control signal that titrates at least one of a second temperature or a flow parameter of the fluid to the anatomic site to manage a temperature of the anatomic site toward a desired target temperature.

17. The method of claim 16, further comprising:
removing, by a suction device, fluid from the anatomic site resulting in removed fluid; and
transferring, by a second irrigation conduit in fluid communication with the suction device, the removed fluid to a waste receptacle;
wherein the first temperature is a temperature of the removed fluid.

18. The method of claim 17, wherein the first temperature is of the fluid at the anatomic site.

19. The method of claim 17, further comprising:
cooling, by a fluid cooler, a first portion of the fluid resulting in cooled fluid; and
providing, by a second irrigation conduit, the cooled fluid;
wherein the control circuitry adjusts, based on the first temperature, a second temperature to which the fluid cooler cools the cooled fluid.

20. The method of claim 17, further comprising providing, by a display electrically coupled to the control circuitry, a view of the first temperature.

21. A therapy delivery system comprising:
a scope configured to provide a view of an anatomic site;
an energy delivery device configured to provide an electrical or optical energy to the anatomic site;
at least one actuated valve;
a first irrigation conduit in fluid communication with a first portion of the fluid and the at least one actuated valve, the first irrigation conduit configured to provide the first portion directly to the actuated valve resulting in direct fluid;
a fluid cooler in fluid communication with a second portion of the fluid and the at least one actuated valve, the fluid cooler configured to cool a second portion of the fluid based on a temperature setting of the fluid cooler resulting in cooled fluid;
a second irrigation conduit in fluid communication with a mixture of the cooled and direct fluid;
a first temperature sensor situated to provide first temperature data associated with the anatomic site; and
control circuitry electrically coupled to the first temperature sensor, the control circuitry configured to receive the first temperature data and provide a control signal that adjusts a state of the at least one actuated valve, the temperature setting, or a combination thereof to manage a temperature of the anatomic site.
